(19) United States
(12) Patent Application Publication
(10) Pub. No.: US 2026/0193266 A1
(43) Pub. Date: Jul. 9, 2026

(54) SYNTHESIS OF OCTAFLUORO PORPHYRINS

(71) Applicant: Laboratory For Synthetic Chemistry And Chemical Biology Limited, Hong Kong (CN)

(72) Inventors: Chi-Ming Che, Hong Kong (CN); Atif Ali, Hong Kong (CN)

(21) Appl. No.: 19/417,636

(22) Filed: Dec. 12, 2025

Related U.S. Application Data
(60) Provisional application No. 63/743,477, filed on Jan. 9, 2025.

(51) Int. Cl. C07D 487/22

(52) U.S. Cl. CPC C07D 487/22

(57) ABSTRACT

Described are a halogenated porphyrin and methods of synthesizing a halogenated porphyrin, including a chiral halogenated porphyrin (e.g., chiral fluorinated porphyrin) and/or a bulkier halogenated porphyrin (e.g., a bulkier fluorinated porphyrin). The halogenated porphyrin can be metalated or non-metalated. A halogenated porphyrin is produced by slowly reacting a halogenated pyrrole and an aryl aldehyde in a reaction catalyzed by boron trifluoride diethyl etherate. Upon formation of a porphyrinogen, the reaction is quenched using triethyl amine and dichloro-5,6-dicyano-1,4-benzoquinone is added to oxidize the porphyrinogen and obtain the halogenated porphyrin. Advantageously, the disclosed methods can be used to synthesize halogenated porphyrins at gram scale, in particular β-octafluorinated porphyrins.

### CROSS-REFERENCE TO RELATED APPLICATIONS

This application claims the benefit of and priority to U.S. Provisional Application No. 63/743,477 filed Jan. 9, 2025, which is herein incorporated by reference in its entirety.

### FIELD OF THE INVENTION

The invention is in the field of halogenated porphyrins (metalated or non-metalated), particularly methods of synthesizing (i) halogenated porphyrins with high yields, (ii) chiral halogenated porphyrins (e.g., chiral β-octafluoro porphyrins), (iii) halogenated porphyrins (e.g., 3-octafluoro porphyrins) with bulky substituents at one or more meso-positions, a combination thereof, and uses of the halogenated porphyrins.

### BACKGROUND OF THE INVENTION

Halogenated porphyrins can be used in several applications, such as in catalytic systems that selectively convert oxygen to water. An example is oxygen reduction reaction that is important in some biological process and technologies involving energy storage. However, the syntheses of halogenated porphyrins are limited to those that contain a less bulky aldehyde such as benzaldehyde or its less bulky derivatives at the meso-positions of porphyrins. Further, to the best of the inventors' knowledge, there are no examples for the synthesis of chiral β-octafluoro porphyrins. It is believed that this absence is due to the poor stability and extreme reactivity of 3,4-difluoropyrrole, a reagent utilized in the synthesis of β-octafluoro porphyrins. These existing methods typically result in low yields and/or quantities that are not relevant for practical applications, such as for commercial purposes. Accordingly, there remains an unmet to develop halogenated porphyrins in yields and/or quantities relevant for practical applications.

It is therefore an object of the invention to provide methods for the improved synthesis of halogenated porphyrins (e.g., β-halogenated porphyrins).

It is also an object of the invention to provide chiral halogenated porphyrins (e.g., chiral (3-halogenated porphyrins) and/or halogenated porphyrins (e.g., β-halogenated porphyrins) containing bulkier substituents at one or more meso-positions of the halogenated porphyrins.

### SUMMARY OF THE INVENTION

Described herein are methods of making a halogenated porphyrin. The disclosed methods can be utilized to synthesize a chiral halogenated porphyrin (e.g., chiral fluorinated porphyrin) and/or a bulkier halogenated porphyrin (e.g., a bulkier fluorinated porphyrin) at gram scale. A zinc-containing halogenated porphyrin can be prepared by deprotonating a corresponding halogenated porphyrin ligand by treating with zinc acetate. A fluorinated porphyrin is produced by combining a halogenated pyrrole (e.g., a fluorinated pyrrole such as a 3,4-difluoropyrrole, a bis(3,4-difluoro-1H-pyrrol-2-yl)methane) and an aryl aldehyde in a reaction vessel over a period of between 2 hours and 6 hours, such as about 3 hours under vigorous stirring (such as between about 1,000 rpm and about 1,200 rpm), and forming a first composition. Preferably, the combination occurs over a period of more than one hour under an inert gas atmosphere (e.g., under argon gas). Preferably, the reaction is catalyzed by boron trifluoride diethyl etherate, and is monitored thin layer chromatography.

Upon formation of a porphyrinogen, the reaction is quenched using triethyl amine and an oxidizing agent, such as dichloro-5,6-dicyano-1,4-benzoquinone, is added to oxidize the porphyrinogen and obtaining the halogenated porphyrin.

Advantageously, the disclosed methods can be used to synthesize halogenated porphyrins at gram scale, in particular β-octafluorinated porphyrins. The following compounds described in the examples, were synthesized at gram scale: 2,3,7,8,12,13,17,18-Octafluoro-5,10,15,20-tetraphenylporphyrin, 2,3,7,8,12,13,17,18-Octafluoro-5,10,15,20-tetrakis(4-methylphenyl)porphyrin, 2,3,7,8,12,13,17,18-Octafluoro-5,10,15,20-tetrakis(2,6-dichloro-4-methylphenyl)porphyrin, 2,3,7,8,12,13,17,18-Octafluoro-5,10,15,20-tetrakis(4-(tert-butyl)-2,6-dichlorophenyl)porphyrin, 2,3,7,8,12,13,17,18-Octafluoro-5,10,15,20-tetrakis(2,6-dibromo-4-(tert-butyl)phenyl)porphyrin, 2,3,7,8,12,13,17,18-Octafluoro-5,10,15,20-tetrakis(4-nitrophenyl)porphyrin, 2,3,7,8,12,13,17,18-Octafluoro-5,10,15,20-tetrakis(3-methyl-4-nitrophenyl)porphyrin.

The disclosed halogenated porphyrins can have a structure:

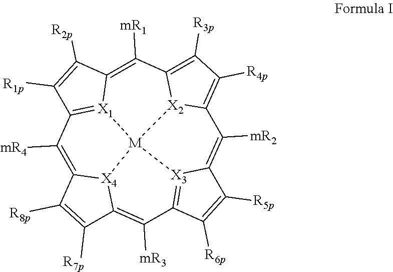

wherein:
M is a transition metal that can be present or absent, and has an oxidation state of 0, +1, +2, +3, +4, +5, +6, or +7,
the dashed lines denote the presence of absence of a bond,
R₁ₚ-R₈ₚ are independently selected from hydrogen, halogen, unsubstituted C₁-C₅ alkyl, substituted C₁-C₅ alkyl, or a combination thereof, wherein at least one of R₁ₚ-R₈ₚ is a halogen,
mR₁-mR₄ are independently selected from aryl, alkylaryl; haloaryl; halo-alkylaryl; octahydro-1,4:5,8-dimethanoanthracene-9-yl; alkyl [1,1′-biaryl]-4-carboxylate; or a combination thereof, wherein mR₁-mR₄ are not all aryl (e.g., phenyl),
X₁-X₄ are nitrogen and independently contain zero, one, or two hydrogen atoms according to valency, and
mR₁-mR₄ are optionally substituted with one or more substituents.

Preferably, mR₁-mR₄ are independently selected from aryl, 2,4,6-trialkylaryl; 2,6-dihaloaryl; 2,6-dihalo-4-alkylaryl; octahydro-1,4:5,8-dimethanoanthracene-9-yl; alkyl [1,1′-biaryl]-4-carboxylate; 4-nitroaryl; 3-alkyl-4-nitroaryl; or a combination thereof. More preferably, mR₁-mR₄ are independently selected from phenyl, 2,4,6-trimethylphenyl, 2,6-dichlorophenyl, 2,6-dichloro-4-methylphenyl, 4-(tert-butyl)-2,6-dichlorophenyl, 2,6-dibromophenyl, 2,6-dibromo-4-(tert-butyl)phenyl, (1S,4R,5R,8S)-1,2,3,4,5,6,7,8-octatahydro-1,4:5,8-dimethanoanthracene-9-yl, methyl [1,1′-biphenyl]-4-carboxylate; 4-nitrophenyl, 3-methyl-4-nitrophenyl, or a combination thereof.

Preferably, R₁ₚ-R₈ₚ are fluorine.

In some forms, M is absent. In other forms, M is present, the dashed lines denote the presence of a bond, and M is a transition metal. In other forms, M is present, the dashed lines denote the presence of a bond, and M is a first-row transition metal (such as a zinc, nickel, etc.) In other forms, M is present, the dashed lines denote the presence of a bond and M is a second-row transition metal or a third-row transition metal (such as platinum).

In some forms, the halogenated compound has a structure:

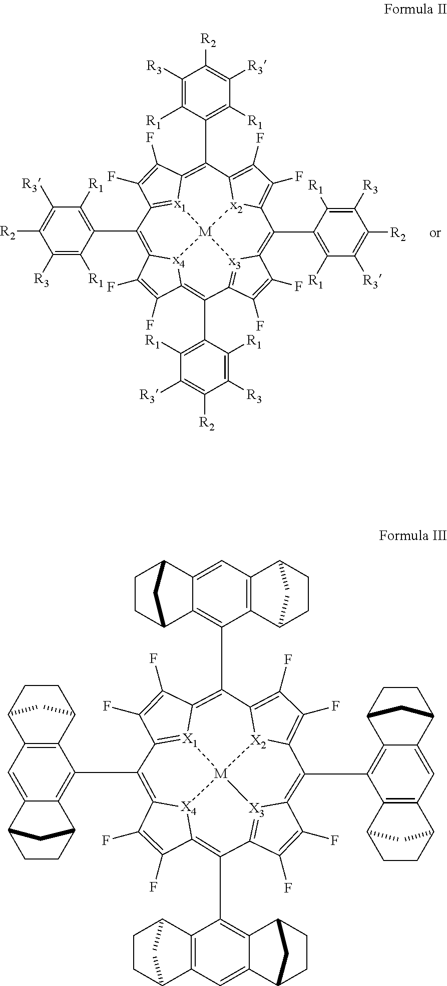

wherein:
for Formula II and Formula III, M is a transition metal that can be present or absent, and has an oxidation state of 0, +1, +2, +3, +4, +5, +6, or +7,
the dashed lines denote the presence of absence of a bond,
X₁-X₄ are nitrogen, and X₁-X₄ independently contain zero, one, or two hydrogen atoms according to valency,
for Formula II:
R₁, R₂, R₃, R₃′=H;
R₁, R₂=—CH₃, R₃, R₃′=H;
R₁=Cl, R₂ R₃, R₃′=H;
R₁=Cl, R₂=—CH₃, R₃, R₃′=H;
R₁=Cl, R₂=-tert-butyl, R₃, R₃=H;
R₁, R₃, R₃′=H, R₂=Br;
R₁=Br, R₂, R₃, R₃′=H;
R₁=Br, R₂=-tert-butyl, R₃, R₃=H;
R₁, R₃, R₃′=H

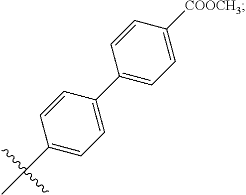 [SMILES: *c1ccc(-c2ccc(C(=O)OC)cc2)cc1]

R₁, R₃, R₃′=H, R₂=R₁, R₃, R₃′=H, R₂=—NO₂; or R₁, R₃, =H, R₂=—NO₂, R₃′=—CH₃.

### DETAILED DESCRIPTION OF THE INVENTION

### I. Definitions

“Bulky substituent,” as relates to a chemical moiety at one or more meso-positions of a halogenated porphyrin, refers to a chemical moiety, such as an aryl group (e.g. phenyl group), that has been modified with heavier atomic mass atoms such as chlorine, methyl, or bulkier groups which make the starting corresponding aldehyde bulky for the reaction with fluorinated pyrrole.

“Chiral halogenated porphyrin” refers to a halogenated porphyrin that has become asymmetric and non-superimposable on its mirror image. Chiral halogenated porphyrin includes porphyrins formed from halogenated porphyrin molecules substituted with one or more chiral groups; and achiral halogenated porphyrins induced by chiral additives and spontaneous deracemizations of achiral halogenated porphyrins during the assemblies.

“Room temperature” refers to temperature from 18° C. to 25° C., such as 22° C. to 25° C.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms.

It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Vigorous stirring” refers to stirring a solution, mixture, etc., at a rate between about 1,000 rpm and about 1,200 rpm.

### II. Compositions

Disclosed are halogenated porphyrins having a structure:

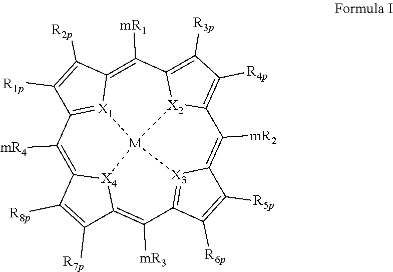

wherein:
M is a transition metal that can be present or absent, and has an oxidation state of 0, +1, +2, +3, +4, +5, +6, or +7,
the dashed lines denote the presence of absence of a bond,
R₁ₚ-R₈ₚ are independently selected from hydrogen, halogen, unsubstituted C₁-C₅ alkyl, substituted C₁-C₅ alkyl, or a combination thereof, wherein at least one of R₁ₚ-R₈ₚ is a halogen,
mR₁-mR₄ are independently selected from aryl, heteroaryl, alkylaryl; alkylheteroaryl; haloaryl; haloheteroaryl; halo-alkylaryl; halo-alkylheteroaryl; octahydro-1,4:5,8-dimethanoanthracene-9-yl; alkyl [1,1′-biaryl]-4-carboxylate; alkyl [1,1′-biheteroaryl]-4-carboxylate; nitroaryl; alkyl-nitroaryl, or a combination thereof, wherein mR₁-mR₄ are not all aryl (e.g., phenyl),
X₁-X₄ are independently nitrogen, oxygen, or sulfur, preferably nitrogen, and X₁-X₄ independently contain zero, one, or two hydrogen atoms according to valency, and
mR₁-mR₄ are optionally substituted with one or more substituents.

In some forms, the halogenated porphyrins are as described above, except that mR₁-mR₄ are independently selected from aryl, alkylaryl; haloaryl; halo-alkylaryl; octahydro-1,4:5,8-dimethanoanthracene-9-yl; alkyl [1,1′-biaryl]-4-carboxylate; nitroaryl; alkyl-nitroaryl, or a combination thereof. In some forms, mR₁-mR₄ are independently selected from aryl, heteroaryl, 2,4,6-trialkylaryl; 2,4,6-trialkylheteroaryl; 2,6-dihaloaryl; 2,6-dihaloheteroaryl; 2,6-dihalo-4-alkylaryl; 2,6-dihalo-4-alkylheteroaryl; octahydro-1,4:5,8-dimethanoanthracene-9-yl; alkyl [1,1′-biaryl]-4-carboxylate; alkyl [1,1′-biheteroaryl]-4-carboxylate; 4-nitroaryl; 3-alkyl-4-nitroaryl; or a combination thereof. In some forms, mR₁-mR₄ are independently selected from aryl, 2,4,6-trialkylaryl; 2,6-dihaloaryl; 2,6-dihalo-4-alkylaryl; octahydro-1,4:5,8-dimethanoanthracene-9-yl; alkyl [1,1′-biaryl]-4-carboxylate; or a combination thereof. In some forms, mR₁-mR₄ are independently selected from phenyl, 2,4,6-trimethylphenyl, 2,6-dichlorophenyl, 2,6-dichloro-4-methylphenyl, 4-(tert-butyl)-2,6-dichlorophenyl, 2,6-dibromophenyl, 2,6-dibromo-4-(tert-butyl)phenyl, (1S,4R,5R,8S)-1,2,3,4,5,6,7,8-octatahydro-1,4:5,8-dimethanoanthracene-9-yl, methyl [1,1′-biphenyl]-4-carboxylate, 4-nitrophenyl; 3-methyl-4-nitrophenyl, or a combination thereof.

In some forms, the halogenated porphyrins are as described above, except that mR₁-mR₄ form a symmetrical substitution pattern. In some forms, the halogenated porphyrins are as described above, except that mR₁-mR₄ form an unsymmetrical substitution pattern.

In some forms, the halogenated porphyrins are as described above, except that at least one of R₁ₚ-R₈ₚ is fluorine. In some forms, R₁ₚ-R₈ₚ are fluorine.

In some forms, the halogenated porphyrins are as described above, except that M is absent. In some forms, the halogenated porphyrins are as described above, except that M is present and the dashed lines denote the presence of a bond. In some forms, M is a first-row transition metal. In some forms, M is a second-row transition metal. In some forms, M is a third-row transition metal. In some forms, M is a zinc. In some forms, M is a nickel. In some forms, M is a platinum.

In some forms, the halogenated compound has a structure:

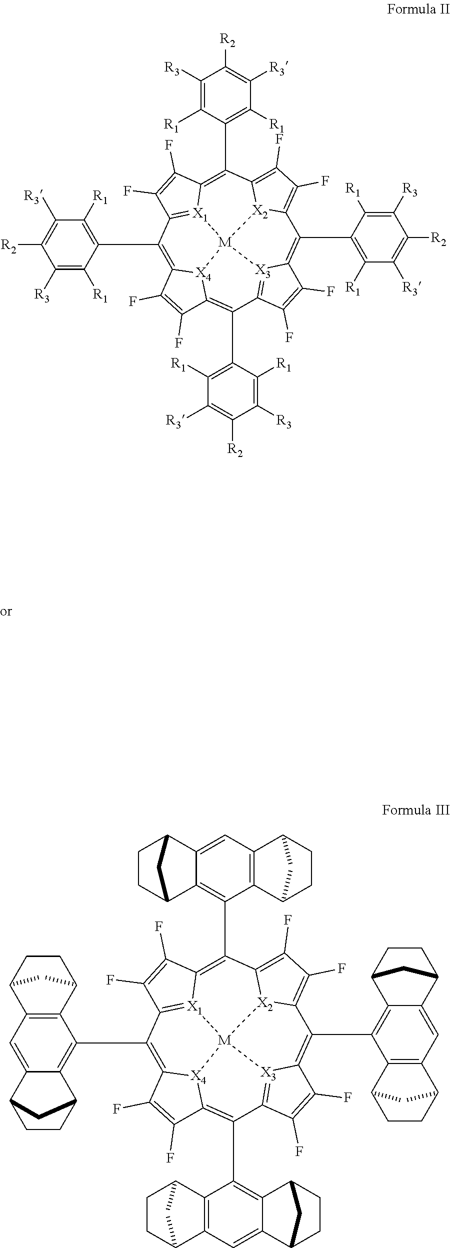

wherein:
for Formula II and Formula III, M is a transition metal that can be present or absent, and has an oxidation state of 0, +1, +2, +3, +4, +5, +6, or +7,
the dashed lines denote the presence of absence of a bond,
X₁-X₄ are nitrogen, and X₁-X₄ independently contain zero, one, or two hydrogen atoms according to valency,
for Formula II:
R₁, R₂, R₃, R₃′=H;
R₁, R₂=—CH₃, R₃, R₃′=H;
R₁=Cl, R₂ R₃, R₃′=H;
R₁=Cl, R₂=—CH₃, R₃, R₃′=H;
R₁=Cl, R₂=-tert-butyl, R₃, R₃=H;
R₁, R₃, R₃′=H, R₂=Br;
R₁=Br, R₂, R₃, R₃′=H;
R₁=Br, R₂=-tert-butyl, R₃, R₃=H;
R₁, R₃, R₃′=H, R₂=

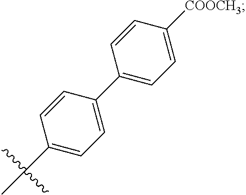 [SMILES: *c1ccc(-c2ccc(C(=O)OC)cc2)cc1]

R₁, R₃, R₃′=H, R₂=—NO₂; or
R₁, R₃, =H, R₂=—NO₂, R₃′=—CH₃.

III. Methods of Making and Reagents therefor Also described are methods of making a halogenated porphyrin. The disclosure provides a method to synthesize a chiral halogenated porphyrin (e.g., chiral fluorinated porphyrin) and/or a bulkier halogenated porphyrin (e.g., a bulkier fluorinated porphyrin). Moreover, a fluorinated-D4-porphyrin can be synthesized by using present method. The designation “D4” denotes molecular symmetry. A metalated halogenated porphyrin can be prepared by deprotonating a corresponding halogenated porphyrin ligand by treating with a salt, such as a metal acetate. A halogenated porphyrin, preferably a fluorinated porphyrin is produced by (i) combining a first reactant and a second reactant in a reaction vessel over a period of between 1 hour and 10 hours, between 1 hour and 8 hours, between 1 hour and 6 hours, between 1 hour and 4 hours, between 1 hour and 3 hours, between 2 hours and 3 hours, between 2 hours and 6 hours, such as over a period of about 3 hours, under stirring (e.g., vigorous stirring, such as between about 1,000 rpm and about 1,200 rpm, or higher), and forming a first composition, and (ii) forming the halogenated porphyrin. Preferably, the first reactant and the second reactant are combined slowly over a period of between 1 hour and 10 hours, between 1 hour and 8 hours, between 1 hour and 6 hours, between 1 hour and 4 hours, between 1 hour and 3 hours, between 2 hours and 3 hours, between 2 hours and 6 hours, such as over a period of about 3 hours. Preferably the first reactant and the second reactant are combined dropwise, where the second reactant is in a reaction vessel and the first reactant is slowly added, or the first reactant is in a reaction vessel and the second reactant is slowly added. The first reactant is a halogenated pyrrole (e.g., a fluorinated pyrrole such as a 3,4-difluoropyrrole, a bis(3,4-difluoro-1H-pyrrol-2-yl)methane), a halogenated furan, a halogenated thiophene, or a combination thereof, and the second reactant is an aryl aldehyde or a heteroaryl aldehyde. A preferred way of combining the first reactant and the second reactant is via dropwise addition in a reaction vessel. Preferably, the first reactant is a halogenated pyrrole (e.g., a fluorinated pyrrole such as a 3,4-difluoropyrrole, a bis(3,4-difluoro-1H-pyrrol-2-yl)methane). Preferably, the methods involve stirring the first composition over a period of more than one hour, over a period greater 1 hour and less than or equal to 10 hours, greater 1 hour and less than or equal to 8 hours, greater 1 hour and less than or equal to 6 hours, greater 1 hour and less than or equal to 4 hours, at room temperature, under an inert gas atmosphere (e.g., under argon gas), or both.

Typically, in the reaction described above, the reaction vessel contains a Lewis acid catalyst. In some forms, the Lewis acid contains a complex between an ether and a boron trihalide. Preferably, the Lewis acid contains a boron trihalide dialkyl etherate, e.g., boron trifluoride diethyl etherate.

The reaction can be monitored using an analytical technique, such as thin layer chromatography for the formation of, for example, a porphyrinogen. Additional steps of the method involve quenching the reaction with an organic base, such as an amine, preferably a trialkyl amine (e.g., triethyl amine) after the formation of a porphyrinogen, adding an oxidizing agent and oxidizing the porphyrinogen, and obtaining the halogenated porphyrin.

In some forms, the method is as described above, except that the halogenated porphyrin is a chiral halogenated porphyrin.

In some forms of the method, the halogenated porphyrin contains bulky substituents at one or more meso-positions of the halogenated porphyrin, wherein the bulky substituents comprise aryl; heteroaryl; alkylaryl; alkylheteroaryl; haloaryl; haloheteroaryl; halo-alkylaryl; halo-alkylheteroaryl; octahydro-1,4:5,8-dimethanoanthracene-9-yl; alkyl [1,1′-biaryl]-4-carboxylate; alkyl [1,1′-biheteroaryl]-4-carboxylate; nitroaryl; alkyl-nitroaryl; or a combination thereof.

In some forms of the method, the halogenated porphyrin contains bulky substituents at one or more meso-positions of the halogenated porphyrin, wherein the bulky substituents comprise aryl, alkylaryl; haloaryl; halo-alkylaryl; octahydro-1,4:5,8-dimethanoanthracene-9-yl; alkyl [1,1′-biaryl]-4-carboxylate; nitroaryl; alkyl-nitroaryl; or a combination thereof.

In some forms of the method, the halogenated porphyrin contains bulky substituents at one or more meso-positions of the halogenated porphyrin, wherein the bulky substituents comprise aryl, 2,4,6-trialkylaryl; 2,4,6-trialkylheteroaryl; 2,6-dihaloaryl; 2,6-dihaloheteroaryl; 2,6-dihalo-4-alkylaryl; 2,6-dihalo-4-alkylheteroaryl; octahydro-1,4:5,8-dimethanoanthracene-9-yl; alkyl [1,1′-biaryl]-4-carboxylate; alkyl [1,1′-biheteroaryl]-4-carboxylate; 4-nitroaryl; 3-alkyl-4-nitroaryl; or a combination thereof.

In some forms of the method, the halogenated porphyrin contains bulky substituents at one or more meso-positions of the halogenated porphyrin, wherein the bulky substituents comprise aryl, 2,4,6-trialkylaryl; 2,6-dihaloaryl; 2,6-dihalo-4-alkylaryl; octahydro-1,4:5,8-dimethanoanthracene-9-yl; alkyl [1,1′-biaryl]-4-carboxylate; 4-nitroaryl; 3-alkyl-4-nitroaryl; or a combination thereof.

In some forms of the method, the halogenated porphyrin contains bulky substituents at one or more meso-positions of the halogenated porphyrin, wherein the bulky substituents are independently selected from phenyl, 2,4,6-trimethylphenyl, 2,6-dichlorophenyl, 2,6-dichloro-4-methylphenyl, 4-(tert-butyl)-2,6-dichlorophenyl, 2,6-dibromophenyl, 2,6-dibromo-4-(tert-butyl)phenyl, (1S,4R,5R,8S)-1,2,3,4,5,6,7,8-octatahydro-1,4:5,8-dimethanoanthracene-9-yl, methyl [1,1′-biphenyl]-4-carboxylate, 4-nitrophenyl, 3-methyl-4-nitrophenyl, or a combination thereof.

In some forms of the method, the halogenated porphyrin contains symmetrical substitution patterns at meso-positions of the halogenated porphyrin. In some forms of the method, the halogenated porphyrin contains unsymmetrical substitution patterns at meso-positions of the halogenated porphyrin.

In some forms of the method, the halogenated porphyrin is a β-fluorinated porphyrin. In more specific forms of the method, the halogenated porphyrin is a β-octafluorinated porphyrin. As a non-limiting example, β-octafluoro porphyrins can be produced by reacting 3,4-difluoropyrrole with variety of bulky and chiral aromatic aldehydes in the presence of boron trifluoride etherate, followed by oxidation. The 3,4-difluoropyrrole can be synthesized by reacting a 3,3,4,4-tetrafluoropyrrolidinium salt, with a base such as potassium tert-butoxide. Bulkier β-octafluoro porphyrins can also be synthesized. Moreover, β-octafluoro-D4-porphyrins can be synthesized by using disclosed methods. Zinc porphyrins can be prepared by deprotonating corresponding 3-octafluoro porphyrin ligands via treatment with zinc acetate. The disclosed methods can also be utilized to synthesize a chiral β-octafluorinated porphyrin and a β-octafluorinated porphyrin with bulkier substituents at the meso-position of the porphyrin ring.

The disclosed methods can be used to synthesize halogenated porphyrins at gram scale, such as at least 2 grams, in particular β-octafluorinated porphyrins. For example, current syntheses of 3-octafluoro porphyrins are limited to less bulky aldehydes such as benzaldehyde or its less bulky derivatives at meso-positions of porphyrins. Also, to the best of the inventors' knowledge there are no reported examples for the synthesis of chiral β-octafluoro porphyrins, and it is believed that this is due to poor stability and extreme reactivity of 3,4-difluoropyrrole. The disclosed methods successfully solved this problem of obtaining chiral and/or bulky β-octafluoro porphyrins. Bulky (3-octafluoro porphyrins were produced in significant yields by slowly reacting 3,4-difluoropyrrole with aldehydes such as 2,4,6-trimethylbenzaldehyde, 2,6-dichlorobenzaldehyde, 2,6-dichloro-4-methylbenzaldehyde, 4-(tert-butyl)-2,6-dichlorobenzaldehyde, 2,6-dibromobenzaldehyde, 2,6-dibromo-4-(tert-butyl)benzaldehyde, 4-bromobenzaldehyde, 4-methylbenzaldehyde, (1S,4R,5R,8S)-1,2,3,4,5,6,7,8-octatahydro-1,4:5,8-dimethanoanthracene-9-carboxaldehyde, 2,6-difluorobenzaldehyde, benzaldehyde, 4-nitrobenzaldehyde, and 3-methyl-4-nitrobenzaldehyde, in the presence of boron trifluoride etherate.

Porphyrins have four meso-positions and eight β-pyrrole positions. In some forms, the disclosed methods incorporate four similar (preferably identical) meso substituents on a porphyrin ring. β-octafluoro porphyrins with different meso aromatic substituents were also synthesized with lower yield. However, this limitation can be addressed by using a halogenated dipyrromethene (e.g., fluorinated dipyrromethene).

One important difference between the disclosed methods and those previously described in the synthesis of porphyrins involves the addition of fluorinated pyrrole to avoid the rapid formation of polypyrroles, when bulkier aldehydes are used for condensation with fluorinated pyrrole. It was discovered that bulky aldehydes need more time to react with pyrroles. Addition of fluorinated pyrrole is important to control the reaction. In the disclosed methods, it was not merely the additional reaction time required, but the slow addition (e.g., dropwise addition) of a reactant over an extended period of time (as described herein), optionally accompanied by vigorous stirring (as described herein). For instance, it was discovered that slow addition of fluorinated pyrrole (e.g., dropwise addition) controlled the reaction and avoided the rapid formation of polypyrrole impurities. The disclosed methods not only increase the yield, they also provide methods to obtained sterically bulky fluorinated porphyrins in gram scale (e.g., at least 2 grams). When previously reported methods were followed, only trace amounts (significantly less than gram-scale quantities) of fluorinated porphyrins were obtained. To the best of the inventors' knowledge, the approach to increase reaction time to synthesize bulky fluorinated porphyrins at gram scale via slow addition (e.g., dropwise addition) of pyrroles, and optionally vigorous stirring, during porphyrins syntheses is a new and improved method that has not been previously reported.

### IV. Methods of Using

The disclosed halogenated porphyrins can be used as catalysts in various reactions such as oxidation, hydroxylation, aziridination and amidation, to obtain high turnover and high yield. Moreover, β-fluorinated porphyrins can also be used as photosensitizers; fluorescent probes; sensors; bioconjugation; cell tracking agents, particularly to visualize the initial steps in cell therapies including delivery, migration, and engraftment; contrast agents in magnetic resonance imaging technologies.

The disclosed systems and methods can be further understood through the following numbered paragraphs.
1. A method of making a halogenated porphyrin, preferably fluorinated porphyrin, the method involving:
   (i) reacting a first reactant and a second reactant in a reaction vessel over a period of between 1 hour and 10 hours, between 1 hour and 8 hours, between 1 hour and 6 hours, between 1 hour and 4 hours, between 1 hour and 3 hours, between 2 hours and 3 hours, between 2 hours and 6 hours, such as over a period of about 3 hours, optionally under stirring (e.g., vigorous stirring between about 1,000 rpm and about 1,200 rpm, or higher), and forming a first composition, wherein the first reactant and the second reactant are combined slowly over a period of between 1 hour and 10 hours, between 1 hour and 8 hours, between 1 hour and 6 hours, between 1 hour and 4 hours, between 1 hour and 3 hours, between 2 hours and 3 hours, between 2 hours and 6 hours, such as over a period of about 3 hours, preferably wherein the first reactant and the second reactant are combined dropwise,
   wherein the first reactant is a halogenated pyrrole (e.g., a fluorinated pyrrole such as a 3,4-difluoropyrrole, a bis(3,4-difluoro-1H-pyrrol-2-yl)methane), a halogenated furan, a halogenated thiophene, or a combination thereof, preferably a halogenated pyrrole (e.g., a fluorinated pyrrole such as a 3,4-difluoropyrrole, a bis(3,4-difluoro-1H-pyrrol-2-yl)methane), and the second reactant is an aryl aldehyde or a heteroaryl aldehyde, and
   (ii) forming the halogenated porphyrin.
2. The method of paragraph 1, further involving after step (i) and before step (ii):
   (ia) stirring the first composition over a period of more than one hour, over a period greater 1 hour and less than or equal to 10 hours, greater 1 hour and less than or equal to 8 hours, greater 1 hour and less than or equal to 6 hours, greater 1 hour and less than or equal to 4 hours, at room temperature, under an inert gas atmosphere (e.g., under argon gas), or both.
3. The method of paragraph 1 or 2, wherein the reaction vessel contains a Lewis acid catalyst.
4. The method of any one of paragraphs 1 to 3, wherein the reaction vessel contains a complex between an ether and a boron trihalide.
5. The method of any one of paragraphs 1 to 4, wherein the reaction vessel contains a boron trihalide dialkyl etherate, e.g., boron trifluoride diethyl etherate.
6. The method of any one of paragraphs 2 to 5, further involving after step (ia) and before step (ii):
   (ib) quenching the reaction with an organic base, such as an amine, preferably a trialkyl amine (e.g., triethyl amine) after the formation of a porphyrinogen.
7. The method of paragraph 6, further involving:
   (ic) adding an oxidizing agent (preferably 2,3-dichloro-5,6-dicyano-1,4-benzoquinone (DDQ)) during or after step (ib), oxidizing the porphyrinogen, and obtaining the halogenated porphyrin.
8. The method of any one of paragraphs 1 to 7, wherein the halogenated porphyrin is a chiral halogenated porphyrin.
9. The method of any one of paragraphs 1 to 8, wherein the halogenated porphyrin contains bulky substituents at one or more meso-positions of the halogenated porphyrin, wherein the bulky substituents contain aryl; heteroaryl; alkylaryl; alkylheteroaryl; haloaryl; haloheteroaryl; halo-alkylaryl; halo-alkylheteroaryl; octahydro-1,4:5,8-dimethanoanthracene-9-yl; alkyl [1,1′-biaryl]-4-carboxylate; alkyl [1,1′-biheteroaryl]-4-carboxylate; nitroaryl; alkyl-nitroaryl; or a combination thereof.
10. The method of any one of paragraphs 1 to 9, wherein the halogenated porphyrin contains bulky substituents at one or more meso-positions of the halogenated porphyrin, wherein the bulky substituents contain aryl, alkylaryl; haloaryl; halo-alkylaryl; octahydro-1,4:5,8-dimethanoanthracene-9-yl; alkyl [1,1′-biaryl]-4-carboxylate; nitroaryl; alkyl-nitroaryl; or a combination thereof.
11. The method of any one of paragraphs 1 to 9, wherein the halogenated porphyrin contains bulky substituents at one or more meso-positions of the halogenated porphyrin, wherein the bulky substituents contain aryl, 2,4,6-trialkylaryl; 2,4,6-trialkylheteroaryl; 2,6-dihaloaryl; 2,6-dihaloheteroaryl; 2,6-dihalo-4-alkylaryl; 2,6-dihalo-4-alkylheteroaryl; octahydro-1,4:5,8-dimethanoanthracene-9-yl; alkyl [1,1′-biaryl]-4-carboxylate; alkyl [1,1′-biheteroaryl]-4-carboxylate; 4-nitroaryl; 3-alkyl-4-nitroaryl; or a combination thereof.
12. The method of any one of paragraphs 1 to 9, wherein the halogenated porphyrin contains bulky substituents at one or more meso-positions of the halogenated porphyrin, wherein the bulky substituents contain aryl, 2,4,6-trialkylaryl; 2,6-dihaloaryl; 2,6-dihalo-4-alkylaryl; octahydro-1,4:5,8-dimethanoanthracene-9-yl; alkyl [1,1′-biaryl]-4-carboxylate; 4-nitroaryl; 3-alkyl-4-nitroaryl; or a combination thereof.
13. The method of any one of paragraphs 1 to 9, wherein the halogenated porphyrin contains bulky substituents at one or more meso-positions of the halogenated porphyrin, wherein the bulky substituents are independently selected from phenyl, 2,4,6-trimethylphenyl, 2,6-dichlorophenyl, 2,6-dichloro-4-methylphenyl, 4-(tert-butyl)-2,6-dichlorophenyl, 2,6-dibromophenyl, 2,6-dibromo-4-(tert-butyl)phenyl, (1S,4R,5R,8S)-1,2,3,4,5,6,7,8-octatahydro-1,4:5,8-dimethanoanthracene-9-yl, methyl [1,1′-biphenyl]-4-carboxylate, 4-nitrophenyl, 3-methyl-4-nitrophenyl, or a combination thereof.
14. The method of any one of paragraphs 1 to 13, wherein halogenated porphyrin contains symmetrical substitution patterns at meso-positions of the halogenated porphyrin.
15. The method of any one of paragraphs 1 to 13, wherein halogenated porphyrin contains unsymmetrical substitution patterns at meso-positions of the halogenated porphyrin.
16. The method of any one of paragraphs 1 to 15, wherein the halogenated porphyrin is a β-fluorinated porphyrin.
17. The method of any one of paragraphs 1 to 16, wherein the halogenated porphyrin is a β-octafluorinated porphyrin.
18. A halogenated porphyrin having a structure:

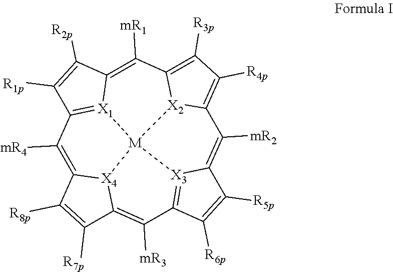

wherein:
M is a transition metal that can be present or absent, and has an oxidation state of 0, +1, +2, +3, +4, +5, +6, or +7,
the dashed lines denote the presence of absence of a bond,
R₁ₚ-R₈ₚ are independently selected from hydrogen, halogen, unsubstituted C₁-C₅ alkyl, substituted C₁-C₅ alkyl, or a combination thereof, wherein at least one of R₁ₚ-R₈ₚ is a halogen,
mR₁-mR₄ are independently selected from aryl, heteroaryl, alkylaryl; alkylheteroaryl; haloaryl; haloheteroaryl; halo-alkylaryl; halo-alkylheteroaryl; octahydro-1,4:5,8-dimethanoanthracene-9-yl; alkyl [1,1′-biaryl]-4-carboxylate; alkyl [1,1′-biheteroaryl]-4-carboxylate; nitroaryl; alkyl-nitroaryl, or a combination thereof, wherein mR₁-mR₄ are not all aryl (e.g., phenyl),
X₁-X₄ are independently nitrogen, oxygen, or sulfur, preferably nitrogen, and X₁-X₄ independently contain zero, one, or two hydrogen atoms according to valency, and
mR₁-mR₄ are optionally substituted with one or more substituents.
19. The halogenated porphyrin of paragraph 18, wherein mR₁-mR₄ are independently selected from aryl, alkylaryl; haloaryl; halo-alkylaryl; octahydro-1,4:5,8-dimethanoanthracene-9-yl; alkyl [1,1′-biaryl]-4-carboxylate; nitroaryl; alkyl-nitroaryl; or a combination thereof.
20. The halogenated porphyrin of paragraph 18, wherein mR₁-mR₄ are independently selected from aryl, heteroaryl, 2,4,6-trialkylaryl; 2,4,6-trialkylheteroaryl; 2,6-dihaloaryl; 2,6-dihaloheteroaryl; 2,6-dihalo-4-alkylaryl; 2,6-dihalo-4-alkylheteroaryl; octahydro-1,4:5,8-dimethanoanthracene-9-yl; alkyl [1,1′-biaryl]-4-carboxylate; alkyl [1,1′-biheteroaryl]-4-carboxylate; 4-nitroaryl; 3-alkyl-4-nitroaryl; or a combination thereof.
21. The halogenated porphyrin of paragraph 18, wherein mR₁-mR₄ are independently selected from aryl, 2,4,6-trialkylaryl; 2,6-dihaloaryl; 2,6-dihalo-4-alkylaryl; octahydro-1,4:5,8-dimethanoanthracene-9-yl; alkyl [1,1′-biaryl]-4-carboxylate; 4-nitroaryl; 3-alkyl-4-nitroaryl; or a combination thereof.
22. The halogenated porphyrin of paragraph 18, wherein mR₁-mR₄ are independently selected from phenyl, 2,4,6-trimethylphenyl, 2,6-dichlorophenyl, 2,6-dichloro-4-methylphenyl, 4-(tert-butyl)-2,6-dichlorophenyl, 2,6-dibromophenyl, 2,6-dibromo-4-(tert-butyl)phenyl, (1S,4R,5R,8S)-1,2,3,4,5,6,7,8-octatahydro-1,4:5,8-dimethanoanthracene-9-yl, methyl [1,1′-biphenyl]-4-carboxylate, 4-nitrophenyl, 3-methyl-4-nitrophenyl, or a combination thereof.
23. The halogenated porphyrin of any one of paragraphs 18 to 22, wherein mR₁-mR₄ form a symmetrical substitution pattern.
24. The halogenated porphyrin of any one of paragraphs 18 to 22, wherein mR₁-mR₄ form an unsymmetrical substitution pattern.
25. The halogenated porphyrin of any one of paragraphs 18 to 24, wherein at least one of R₁ₚ-R₈ₚ is fluorine.
26. The halogenated porphyrin of any one of paragraphs 18 to 25, wherein R₁ₚ-R₈ₚ are fluorine.
27. The halogenated porphyrin of any one of paragraphs 18 to 26, wherein M is present and the dashed lines denote the presence of a bond.
28. The halogenated porphyrin of any one of paragraphs 18 to 27, wherein M is a first-row transition metal, a second-row transition metal, or a third-row transition metal.
29. The halogenated porphyrin of any one of paragraphs 18 to 27, wherein M is a zinc, a nickel, or a platinum.
30. The halogenated porphyrin of any one of paragraphs 18 to 26, wherein M is absent.
31. A halogenated porphyrin, having a structure:

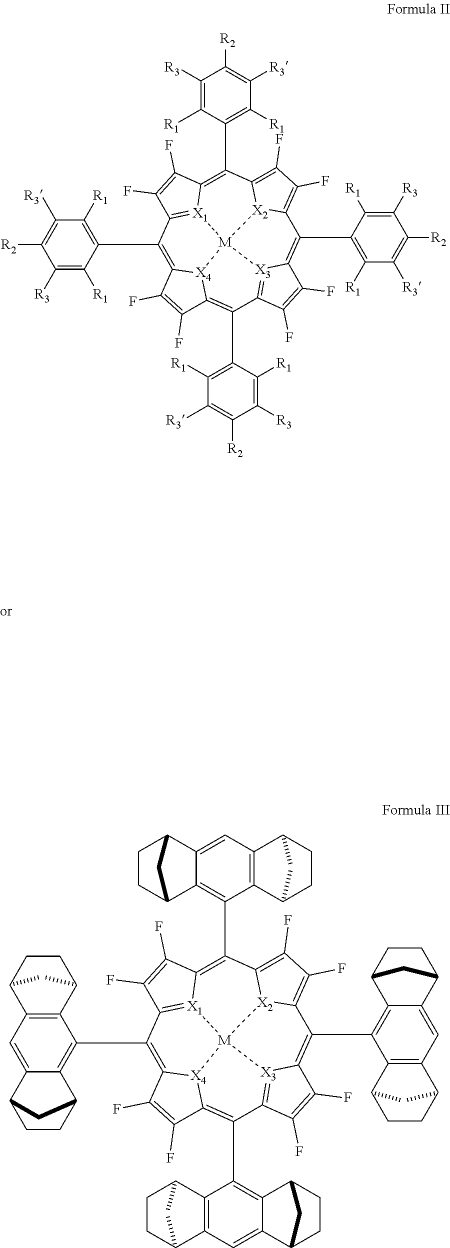

wherein:
for Formula II and Formula III, M is a transition metal that can be present or absent, and has an oxidation state of 0, +1, +2, +3, +4, +5, +6, or +7,
the dashed lines denote the presence of absence of a bond,
X₁-X₄ are nitrogen, and X₁-X₄ independently contain zero, one, or two hydrogen atoms according to valency,
for Formula II:
R₁, R₂, R₃, R₃′=H;
R₁, R₂=—CH₃, R₃, R₃′=H;
R₁=Cl, R₂ R₃, R₃′=H;
R₁=Cl, R₂=—CH₃, R₃, R₃′=H;
R₁=Cl, R₂=-tert-butyl, R₃, R₃=H;
R₁, R₃, R₃′=H, R₂=Br;
R₁=Br, R₂, R₃, R₃′=H;
R₁=Br, R₂=-tert-butyl, R₃, R₃=H;
R₁, R₃, R₃=H, R₂=

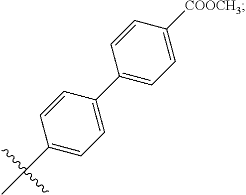 [SMILES: *c1ccc(-c2ccc(C(=O)OC)cc2)cc1]

R₁, R₃, R₃′=H, R₂=—NO₂; or
R₁, R₃, =H, R₂=—NO₂, R₃′=—CH₃.
32. A composition containing the halogenated porphyrin of any one of paragraphs 18 to
31, wherein the halogenated porphyrin is synthesized according to the method of any one of paragraphs 1 to 17.

The methods, compounds, and compositions herein described are further illustrated in the following examples, which are provided by way of illustration and are not intended to be limiting. It will be appreciated that variations in proportions and alternatives in elements of the components shown will be apparent to those skilled in the art and are within the scope of disclosed forms. All parts or amounts, unless otherwise specified, are by weight.

### Examples

### Example 1: Synthesis of p-Octafluoro Porphyrins

### Materials and Methods

### General Procedure for the Synthesis of β-Octafluoro Porphyrins

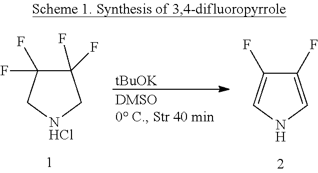

3,3,4,4-Tetrafluoropyrrolidinium chloride (1) (1.0 equiv) was placed in a round-bottom Schlenk flask equipped with a magnetic stirring bar and dissolved in dry DMSO 5 mL at room temperature. The reaction mixture was cooled to 0° C. in a cold-water bath, followed by slow addition of potassium tert-butoxide (4.0 equiv) under an argon using powder addition funnel. The mixture was stirred for 40 minutes at room temperature, then cooled to 0° C. and quenched by addition of ice. The reaction mixture was extracted three times with minimum amount of dichloromethane. The combined organic layers were washed four times with water and twice with brine and dried over magnesium sulfate.

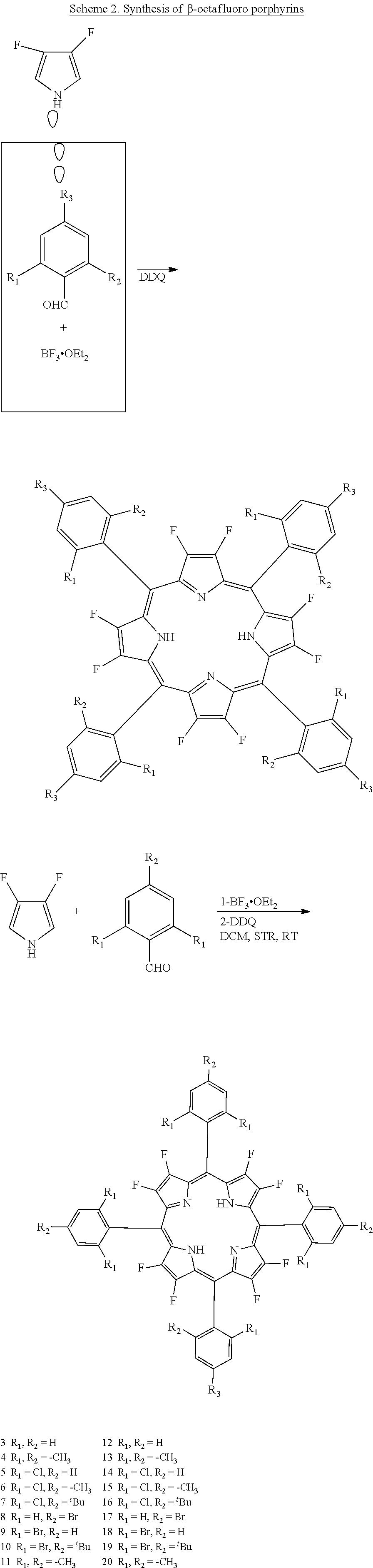

The corresponding aldehyde (1.1 equiv) was added to a round bottom flask equipped with magnetic stirrer bar and diluted with dry dichloromethane until the concentration was 0.01 M. This was followed by addition of boron trifluoride diethyl etherate. 3,4-difluoropyrrole in dichloromethane was added dropwise to reaction mixture over a period of three hours under vigorous stirring. The reaction mixture was further stirred for one more hour at room temperature under argon. The progress of reaction is monitored by thin layer chromatography. Reaction was quenched with addition of triethylamine and 2,3-dichloro-5,6-dicyano-1,4-benzoquinone (DDQ). Reaction mixture was stirred for one hour. The suspension was filtered through a short silica gel column and rinsed with dichloromethane until the eluent became colorless. The obtained crude product was further purified by washing with 10 mL of hexane. Polypyrrole was removed by washing the crude product with methanol until the purified p-octafluoro porphyrin was obtained. p-octafluoro porphyrin was further purified by crystallization from methanol.

### General Procedure for the Metalation of β-Octafluoro Porphyrins

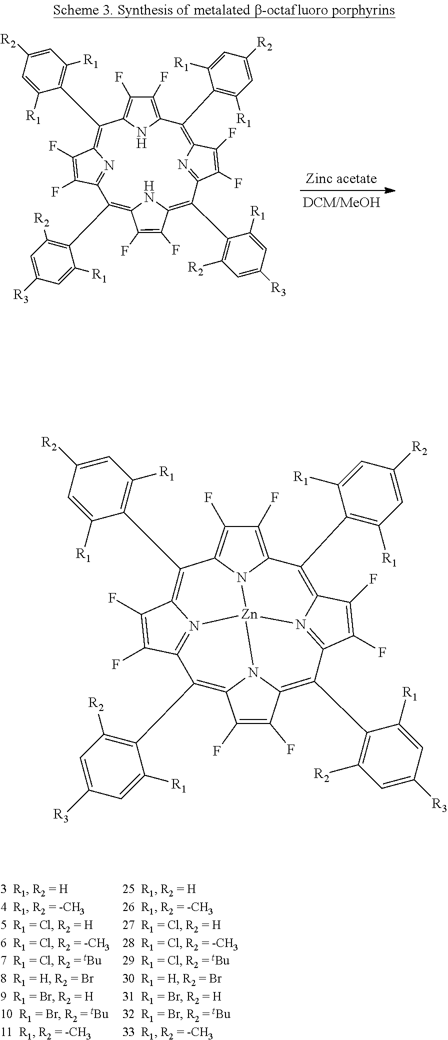

The β-octafluoro porphyrin (50 mg) was suspended in 20 mL of dichloromethane and few drops of methanol solution containing a 10-fold excess of zinc acetate. Progress of reaction was monitored by thin layer chromatography. Metallation was completed in a few hours. The solvent was evaporated and crude product was purified in a short silica gel column using dichloromethane.

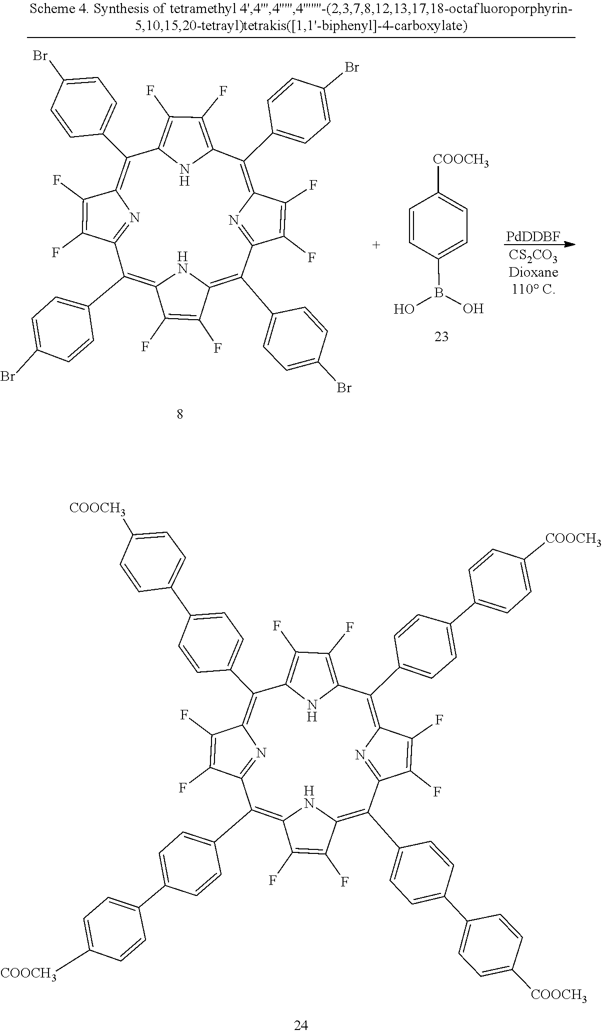

¹H NMR (600 MHz, Chloroform-d) δ 8.24 (d, J=7.7 Hz, 8H), 8.14 (d, J=7.5 Hz, 8H), 7.99 (d, J=7.7 Hz, 16H), 4 (s, 12H), -4.18 (s, 2H)); ¹⁹F NMR (600 MHz, CDCl₃) δ -140.24 (s, 4F), -145.66 (s, 4F).

### Results

### Preparation of 2,3, 7,8,12,13,17,18-Octafluoro-5,10,15,20-tetraphenylporphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

Benzaldehyde 0.65 g (6.17 mM) was added to a round bottom flask equipped with magnetic stirrer bar and diluted with 615 mL of dry dichloromethane, followed by addition of boron trifluoride diethyl etherate 2.48 mL. 3,4-difluoropyrrole in dichloromethane was added dropwise to reaction mixture over a period of three hours under vigorous stirring, reaction mixture was stirred for an additional one hour and progress of reaction was monitored by thin layer chromatography. Reaction was quenched with addition of triethylamine, followed by addition of DDQ to oxidize the porphyrinogen and stirred for one hour. The reaction mixture was purified by washing with 10 mL of hexane and multiple times washed with methanol until the purified 2,3,7,8,12,13,17,18-Octafluoro-5,10,15,20-tetraphenylporphyrin was obtained in 36% yield. 2,3,7,8,12,13,17,18-Octafluoro-5,10,15,20-tetraphenylporphyrin was further purified by crystallization from methanol. The product is characterized by following data: ¹H NMR (600 MHz, CDCl₃) δ 8.07 (d, J=7.4 Hz, 4H), 7.81 (t, J=7.5 Hz, 4H), 7.75 (t, J=7.5 Hz 8H), -4.13 (s, 2H); ¹⁹F NMR (600 MHz, CDCl₃) δ-140.55 (s, 4F), -145.85 (s, 4F); mass: 1.42 g.

### Preparation of 2,3, 7,8,12,13,17,18-Octafluoro-5,10,15,20-tetrakis(4-methylphenyl)porphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

4-Methylbenzaldehyde 0.74 g (6.17 mM) was added to another round bottom flask equipped with magnetic stirrer bar and diluted with 615 mL of dry dichloromethane. Followed by addition of boron trifluoride diethyl etherate 2.48 mL. 3,4-difluoropyrrole in dichloromethane was added dropwise to reaction mixture over a period of three hours under vigorous stirring. The reaction mixture was stirred for one more hour at room temperature under argon. The progress of reaction is monitored by thin layer chromatography. Reaction was quenched with addition of triethylamine and DDQ were added to oxidize the porphyrinogen. Reaction mixture was stirred for one hour. The reaction mixture was purified by washing with 10 mL of hexane. Polypyrrole was removed by washing the crude product with methanol until the purified 2,3,7,8,12,13,17,18-Octafluoro-5,10,15,20-tetrakis(4-methylphenyl)porphyrin was obtained in 36% yield. 2,3,7,8,12,13,17,18-Octafluoro-5,10,15,20-tetrakis(4-methylphenyl)porphyrin was further purified by crystallization from methanol. The product is characterized by following data: ¹H NMR (600 MHz, CDCl₃) δ 7.93 (d, J=7.9 Hz, 8H), 7.53 (d, J=7.7 Hz, 8H), -4.15 (s, 2H); ¹⁹F NMR (600 MHz, CDCl₃) δ -140.23 (s, 4F), -145.67 (s, 4F); mass: 1.04 g.

### Preparation of 2,3, 7,8,12,13,17,18-Octafluoro-5,10,15,20-tetrakis(2, 6-dichlorophenyl)porphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

2,6-Dichlorobenzaldehyde 1.08 g (6.17 mM) was added to another round bottom flask equipped with magnetic stirrer bar and diluted with 620 mL of dry dichloromethane. Followed by addition of boron trifluoride diethyl etherate 2.48 mL. 3,4-difluoropyrrole in dichloromethane was added dropwise to reaction mixture over a period of three hours under vigorous stirring. The reaction mixture was stirred for one more hour at room temperature under argon. The progress of reaction is monitored by thin layer chromatography. Reaction was quenched with addition of triethylamine and DDQ were added to oxidize the porphyrinogen. Reaction mixture was stirred for one hour. The reaction mixture was purified by washing with 10 mL of hexane. Polypyrrole was removed by washing the crude product with methanol until the purified 2,3,7,8,12,13,17,18-Octafluoro-5,10,15,20-tetrakis(2,6-dichlorophenyl)porphyrin was obtained in 36% yield. 2,3,7,8,12,13,17,18-Octafluoro-5,10,15,20-tetrakis(2,6-dichlorophenyl)porphyrin was further purified by crystallization from methanol. The product is characterized by following data: ¹H NMR (600 MHz, CDCl₃) δ 7.77 (d, J=8.4 Hz, 8H), 7.69 (t, J=8.2 Hz, 4H), -3.96 (s, 2H); ¹⁹F NMR (600 MHz, CDCl₃) δ -144.63 (s, 4F), -149.55 (s, 4F).

### Preparation of 2, 3, 7,8,12,13,17,18-Octafluoro-5,10,15, 20-tetrakis(2, 6-dichloro-4-methylphenyl)porphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

2,6-dichloro-4-methylbenzaldehyde 1.16 g (6.17 mM) was added to another round bottom flask equipped with magnetic stirrer bar and diluted with 615 mL of dry dichloromethane. Followed by addition of boron trifluoride diethyl etherate 2.48 mL. 3,4-difluoropyrrole in dichloromethane was added dropwise to reaction mixture over a period of three hours under vigorous stirring. The reaction mixture was stirred for one more hour at room temperature under argon. The progress of reaction is monitored by thin layer chromatography. Reaction was quenched with addition of triethylamine and DDQ were added to oxidize the porphyrinogen. Reaction mixture was stirred for one hour. The reaction mixture was purified by washing with 10 mL of hexane. Polypyrrole was removed by washing the crude product with methanol until the purified 2,3,7,8,12,13,17,18-Octafluoro-5,10,15,20-tetrakis(2,6-dichloro-4-methylphenyl)porphyrin was obtained in 36% yield. 2,3,7,8,12,13,17,18-Octafluoro-5,10,15,20-tetrakis(2,6-dichloro-4-methylphenyl)porphyrin was further purified by crystallization from methanol. The product is characterized by following data: ¹H NMR (600 MHz, CDCl₃) δ 7.54 (s, 8H), 2.57 (s, 12H), -4.07 (s, 2H); ¹⁹F NMR (600 MHz, CDCl₃) δ-145.34 (s, 4F), -150.48 (s, 4F); mass: 1.11 g.

### Preparation of 2, 3, 7,8,12,13,17,18-Octafluoro-5,10,15, 20-tetrakis(4-(tert-butyl)-2, 6-dichlorophenyl)porphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

4-(Tert-butyl)-2,6-dichlorobenzaldehyde 1.42 g (6.17 mM) was added to another round bottom flask equipped with magnetic stirrer bar and diluted with 615 mL of dry dichloromethane. Followed by addition of boron trifluoride diethyl etherate 2.48 mL. 3,4-difluoropyrrole in dichloromethane was added dropwise to reaction mixture over a period of three hours under vigorous stirring. The reaction mixture was stirred for one more hour at room temperature under argon. The progress of reaction is monitored by thin layer chromatography. Reaction was quenched with addition of triethylamine and DDQ were added to oxidize the porphyrinogen. Reaction mixture was stirred for one hour. The reaction mixture was purified by washing with 10 mL of hexane. Polypyrrole was removed by washing the crude product with methanol until the purified 2,3,7,8,12,13,17,18-Octafluoro-5,10,15,20-tetrakis(4-(tert-butyl)-2,6-dichlorophenyl)porphyrin was obtained in 36% yield. 2,3,7,8,12,13,17,18-Octafluoro-5,10,15,20-tetrakis(4-(tert-butyl)-2,6-dichlorophenyl)porphyrin was further purified by crystallization from methanol. The product is characterized by following data: ¹H NMR (600 MHz, CDCl₃) δ 7.74 (s, 8H), 1.56 (s, 36H), -3.96 (s, 2H); ¹⁹F NMR (600 MHz, CDCl₃) δ-144.96 (s, 4F), -150.09 (s, 4F); mass: 1.65 g.

### Preparation of 2,3, 7,8,12,13,17,18-Octafluoro-5,10,15,20-tetrakis(4-bromophenyl)porphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

4-Bromobenzaldehyde 1.14 g (6.17 mM) was added to another round bottom flask equipped with magnetic stirrer bar and diluted with 615 mL of dry dichloromethane. Followed by addition of boron trifluoride diethyl etherate 2.48 mL. 3,4-difluoropyrrole in dichloromethane was added dropwise to reaction mixture over a period of three hours under vigorous stirring. The reaction mixture was stirred for one more hour at room temperature under argon. The progress of reaction is monitored by thin layer chromatography. Reaction was quenched with addition of triethylamine and DDQ were added to oxidize the porphyrinogen. Reaction mixture was stirred for one hour. The reaction mixture was purified by washing with 10 mL of hexane. Polypyrrole was removed by washing the crude product with methanol until the purified 2,3,7,8,12,13,17,18-Octafluoro-5,10,15,20-tetrakis(4-bromophenyl)porphyrin was obtained in 36% yield. 2,3,7,8,12,13,17,18-Octafluoro-5,10,15,20-tetrakis(4-bromophenyl)porphyrin was further purified by crystallization from methanol. The product is characterized by following data: ¹H NMR (600 MHz, CDCl₃) δ 7.87 (m, 8H), -4.24 (s, 2H); ¹⁹F NMR (600 MHz, CDCl₃) δ-140.05 (s, 4F), -144.55 (s, 4F).

### Preparation of 2,3, 7,8,12,13,17,18-Octafluoro-5,10,15,20-tetrakis(2, 6-dibromophenyl)porphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

2,6-Dibromobenzaldehyde 1.62 g (6.17 mM) was added to another round bottom flask equipped with magnetic stirrer bar and diluted with 620 mL of dry dichloromethane. Followed by addition of boron trifluoride diethyl etherate 2.48 mL. 3,4-difluoropyrrole in dichloromethane was added dropwise to reaction mixture over a period of three hours under vigorous stirring. The reaction mixture was stirred for one more hour at room temperature under argon. The progress of reaction is monitored by thin layer chromatography. Reaction was quenched with addition of triethylamine and DDQ were added to oxidize the porphyrinogen. Reaction mixture was stirred for one hour. The reaction mixture was purified by washing with 10 mL of hexane. Polypyrrole was removed by washing the crude product with methanol until the purified 2,3,7,8,12,13,17,18-Octafluoro-5,10,15,20-tetrakis(2,6-dibromophenyl)porphyrin was obtained in 36% yield. 2,3,7,8,12,13,17,18-Octafluoro-5,10,15,20-tetrakis(2,6-dibromophenyl)porphyrin was further purified by crystallization from methanol. The product is characterized by following data: ¹H NMR (600 MHz, CDCl₃) δ 8.00 (d, J=8.28 Hz, 8H), 7.69 (t, J=8.27 Hz, 4H), -3.84 (s, 2H); ¹⁹F NMR (600 MHz, CDCl₃) δ-144.18 (s, 4F), -149.36 (s, 4F).

### Preparation of 2, 3, 7,8,12,13,17,18-Octafluoro-5,10,15, 20-tetrakis(2, 6-dibromo-4-(tert-butyl)phenyl)porphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

2,6-Dibromo-4-(tert-butyl)benzaldehyde 1.97 g (6.17 mM) was added to another round bottom flask equipped with magnetic stirrer bar and diluted with 620 mL of dry dichloromethane. Followed by addition of boron trifluoride diethyl etherate 2.48 mL. 3,4-difluoropyrrole in dichloromethane was added dropwise to reaction mixture over a period of three hours under vigorous stirring. The reaction mixture was stirred for one more hour at room temperature under argon. The progress of reaction is monitored by thin layer chromatography. Reaction was quenched with addition of triethylamine and DDQ were added to oxidize the porphyrinogen. Reaction mixture was stirred for one hour. The reaction mixture was purified by washing with 10 mL of hexane. Polypyrrole was removed by washing the crude product with methanol until the purified 2,3,7,8,12,13,17,18-Octafluoro-5,10,15,20-tetrakis(2,6-dibromo-4-(tert-butyl)phenyl)porphyrin was obtained in 36% yield. 2,3,7,8,12,13,17,18-Octafluoro-5,10,15,20-tetrakis(2,6-dibromo-4-(tert-butyl)phenyl)porphyrin was further purified by crystallization from methanol. The product is characterized by following data: ¹H NMR (600 MHz, CDCl₃) δ 7.96 (s, 8H), 1.55 (s, 36H), -3.83 (s, 2H); ¹⁹F NMR (600 MHz, CDCl₃) δ-144.50 (s, 4F), -149.88 (s, 4F); mass: 1.38 g.

### Preparation of 2,3, 7,8,12,13,17,18-Octafluoro-5,10,15,20-tetramesitylporphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

Mesitylaldehyde 0.91 g (6.17 mM) was added to another round bottom flask equipped with magnetic stirrer bar and diluted with 615 mL of dry dichloromethane. Followed by addition of boron trifluoride diethyl etherate 2.48 mL. 3,4-difluoropyrrole in dichloromethane was added dropwise to reaction mixture over a period of three hours under vigorous stirring. The reaction mixture was stirred for one more hour at room temperature under argon. The progress of reaction is monitored by thin layer chromatography. Reaction was quenched with addition of triethylamine and DDQ were added to oxidize the porphyrinogen. Reaction mixture was stirred for one hour. The reaction mixture was purified by washing with 10 mL of hexane. Polypyrrole was removed by washing the crude product with methanol until the purified 2,3,7,8,12,13,17,18-Octafluoro-5,10,15,20-tetramesitylporphyrin was obtained in 36% yield. 2,3,7,8,12,13,17,18-Octafluoro-5,10,15,20-tetramesitylporphyrin was further purified by crystallization from methanol. The product is characterized by following data: ¹H NMR (600 MHz, CDCl₃) δ 7.27 (s, 8H), 2.26 (s, 12H), 1.90 (s, 24H), -3.91 (s, 2H); ¹⁹F NMR (600 MHz, CDCl₃) δ-144.25 (s, 4F), -149.50 (s, 4F).

### Preparation of 2,3, 7,8,12,13,17,18-Octafluoro-5,10,15,20-tetrakis[(1S,4R,5R,8S)-1,2,3,4,5,6,7,8-octatahydro-1,4:5,8-dimethanoanthracene-9-yl]-porphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

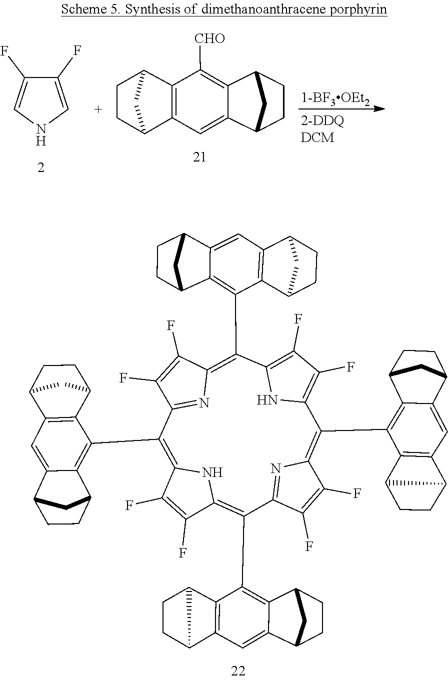

(1S,4R,5R,8S)-1,2,3,4,5,6,7,8-Octatahydro-1,4:5,8-dimethanoanthracene-9-carboxaldehyde 1.46 g (6.17 mM) was added to another round bottom flask equipped with magnetic stirrer bar and diluted with 615 mL of dry dichloromethane. Followed by addition of boron trifluoride diethyl etherate 2.48 mL. 3,4-difluoropyrrole in dichloromethane was added dropwise to reaction mixture over a period of three hours under vigorous stirring. The reaction mixture was stirred for one more hour at room temperature under argon. The progress of reaction is monitored by thin layer chromatography. Reaction was quenched with addition of triethylamine and DDQ were added to oxidize the porphyrinogen. Reaction mixture was stirred for one hour. The reaction mixture was purified by washing with 10 mL of hexane. Polypyrrole was removed by washing the crude product with methanol until the purified 2,3,7,8,12,13,17,18-Octafluoro-5,10,15,20-tetrakis[(1S,4R,5R,8S)-1,2,3,4,5,6,7,8-octatahydro-1,4:5,8-dimethanoanthracene-9-yl]-porphyrin was obtained in 36% yield. 2,3,7,8,12,13,17,18-Octafluoro-5,10,15,20-tetrakis[(1S,4R,5R,8S)-1,2,3,4,5,6,7,8-octatahydro-1,4:5,8-dimethanoanthracene-9-yl]-porphyrin was further purified by crystallization from methanol. ¹H NMR (600 MHz, CDCl₃) δ 7.36 (s, 4H), 3.55 (d, J=3.55 Hz, 8H), 2.69 (s, 8H), 1.99 (d, J=8.0 Hz, 24H), 1.90 (m, 8H), 1.41 (m, 24H), 0.95 (m, 8H) -3.85 (s, 2H); ¹⁹F NMR (600 MHz, CDCl₃) δ-142.52 (s, 4F), -148.53 (s, 4F).

### Preparation of [2, 3, 7,8,12,13,17,18-octafluoro-5,10,15,20-tetraphenylporphinato]zinc

2,3,7,8,12,13,17,18-octafluoro-5,10,15,20-tetraphenylporphyrin (50 mg) was suspended in 20 mL of dichloromethane and few drops of methanol solution containing a zinc acetate 150 mg was added. Progress of reaction was monitored by thin layer chromatography. Metallation of 2,3,7,8,12,13,17,18-octafluoro-5,10,15,20-tetraphenylporphyrin was completed in 2 hours. The solvent was evaporated and metal complex was purified short silica gel column using dichloromethane.

### Preparation of [2,3, 7,8,12,13,17,18-Octafluoro-5,10,15,20-tetrakis(4-methylphenyl)porphinato]zinc

2,3,7,8,12,13,17,18-Octafluoro-5,10,15,20-tetrakis(4-methylphenyl)porphyrin (50 mg) was suspended in 20 mL of dichloromethane and drops of methanol solution containing a zinc acetate 150 mg was added. Progress of reaction was monitored by thin layer chromatography. Metallation of 2,3,7,8,12,13,17,18-Octafluoro-5,10,15,20-tetrakis(4-methylphenyl)porphyrin was completed in 2 hours. The solvent was evaporated and metal complex was purified short silica gel column using dichloromethane.

### Preparation of [2,3, 7,8,12,13,17,18-Octafluoro-5,10,15,20-tetrakis(2, 6-dichlorophenyl)porphinato]zinc

2,3,7,8,12,13,17,18-Octafluoro-5,10,15,20-tetrakis(2,6-dichlorophenyl)porphyrin (50 mg) was suspended in 20 mL of dichloromethane and few drops of methanol solution containing a zinc acetate 150 mg was added. Progress of reaction was monitored by thin layer chromatography. Metallation of 2,3,7,8,12,13,17,18-Octafluoro-5,10,15,20-tetrakis(2,6-dichlorophenyl)porphyrin was completed in 12 hours. The solvent was evaporated and metal complex was purified short silica gel column using dichloromethane.

### Preparation of [2,3, 7,8,12,13,17,18-Octafluoro-5,10,15,20-tetrakis(2, 6-dichloro-4-methylphenyl)porphinato]zinc

2,3,7,8,12,13,17,18-Octafluoro-5,10,15,20-tetrakis(2,6-dichloro-4-methylphenyl)porphyrin (50 mg) was suspended in 20 mL of dichloromethane and few drops of methanol solution containing a zinc acetate 150 mg was added. Progress of reaction was monitored by thin layer chromatography. Metallation of 2,3,7,8,12,13,17,18-Octafluoro-5,10,15,20-tetrakis(2,6-dichloro-4-methylphenyl)porphyrin was completed in 12 hours. The solvent was evaporated and metal complex was purified short silica gel column using dichloromethane.

### Preparation of [2, 3, 7,8,12,13,17,18-Octafluoro-5,10,15,20-tetrakis(4-(tert-butyl)-2,6-dichlorophenyl)porphinato]zinc

2,3,7,8,12,13,17,18-Octafluoro-5,10,15,20-tetrakis(4-(tert-butyl)-2,6-dichlorophenyl)porphyrin (50 mg) was suspended in 20 mL of dichloromethane and few drops of methanol solution containing a zinc acetate 150 mg was added. Progress of reaction was monitored by thin layer chromatography. Metallation of 2,3,7,8,12,13,17,18-Octafluoro-5,10,15,20-tetrakis(4-(tert-butyl)-2,6-dichlorophenyl)porphyrin was completed in 6 hours. The solvent was evaporated and metal complex was purified short silica gel column using dichloromethane.

### Preparation of [2,3, 7,8,12,13,17,18-Octafluoro-5,10,15,20-tetrakis(4-bromophenyl)porphinato]zinc

2,3,7,8,12,13,17,18-Octafluoro-5,10,15,20-tetrakis(4-bromophenyl)porphyrin (50 mg) was suspended in 20 mL of dichloromethane and few drops of methanol solution containing a zinc acetate 150 mg was added. Progress of reaction was monitored by thin layer chromatography. Metallation of 2,3,7,8,12,13,17,18-Octafluoro-5,10,15,20-tetrakis(4-bromophenyl)porphyrin was completed in 2 hours. The solvent was evaporated and metal complex was purified short silica gel column using dichloromethane.

### Preparation [2,3, 7,8,12,13,17,18-Octafluoro-5,10,15,20-tetrakis(2, 6-dibromophenyl)porphinato]zinc

2,3,7,8,12,13,17,18-Octafluoro-5,10,15,20-tetrakis(2,6-dibromophenyl)porphyrin (50 mg) was suspended in 20 mL of dichloromethane and few drops of methanol solution containing a zinc acetate 150 mg was added. Progress of reaction was monitored by thin layer chromatography. Metallation of 2,3,7,8,12,13,17,18-Octafluoro-5,10,15,20-tetrakis(2,6-dibromophenyl)porphyrin was completed in 12 hours. The solvent was evaporated and metal complex was purified short silica gel column using dichloromethane.

### Preparation of[2,3, 7,8,12,13,17,18-Octafluoro-5,10,15,20-tetrakis(2, 6-dibromo-4-(tert-butyl)phenyl)porphinato]zinc

2,3,7,8,12,13,17,18-Octafluoro-5,10,15,20-tetrakis(2,6-dibromo-4-(tert-butyl)phenyl)porphyrin (50 mg) was suspended in 20 mL of dichloromethane and few drops of methanol solution containing a zinc acetate 150 mg was added. Progress of reaction was monitored by thin layer chromatography. Metallation of 2,3,7,8,12,13,17,18-Octafluoro-5,10,15,20-tetrakis(2,6-dibromo-4-(tert-butyl)phenyl)porphyrin was completed in 12 hours. The solvent was evaporated and metal complex was purified short silica gel column using dichloromethane.

### Preparation of [2,3, 7,8,12,13,17,18-Octafluoro-5,10,15,20-tetrakis[(S,4R,5R,8S)-1,2,3,4,5,6,7,8-octatahydro-1,4:5,8-dimethanoanthracene-9-yl]-porphinato]zinc

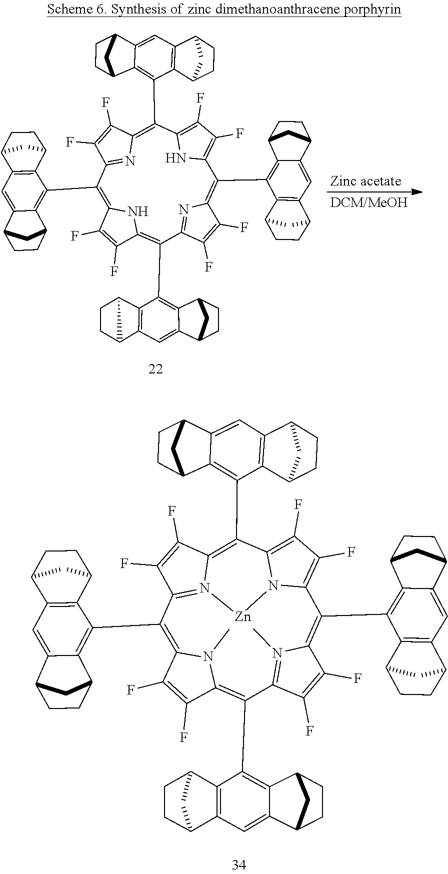

2,3,7,8,12,13,17,18-Octafluoro-5,10,15,20-tetrakis[(1S,4R,5R,8S)-1,2,3,4,5,6,7,8-octatahydro-1,4:5,8-dimethanoanthracene-9-yl]-porphyrin (50 mg) was suspended in 20 mL of dichloromethane and few drops of methanol solution containing a zinc acetate 150 mg was added. Progress of reaction was monitored by thin layer chromatography. Metallation of 2,3,7,8,12,13,17,18-Octafluoro-5,10,15,20-tetrakis[(1S,4R,5R,8S)-1,2,3,4,5,6,7,8-octatahydro-1,4:5,8-dimethanoanthracene-9-yl]-porphyrin was completed in 2 hours. The solvent was evaporated and metal complex was purified short silica gel column using dichloromethane.

### Preparation of [2, 3, 7,8,12,13,17,18-Octafluoro-5,10,15,20-tetrakis(4-(tert-butyl)-2,6-dichlorophenyl)porphinato]nickel

2,3,7,8,12,13,17,18-Octafluoro-5,10,15,20-tetrakis(4-(tert-butyl)-2,6-dichlorophenyl)porphyrin (50 mg) and anhydrous nickel acetate 4 equivalent (28 mg) was suspended in 20 mL of N,N-dimethylformamide. The reaction mixture was refluxed and progress of reaction was monitored by thin layer chromatography. Metallation of 2,3,7,8,12,13,17,18-octafluoro-5,10,15,20-tetrakis(4-(tert-butyl)-2,6-dichlorophenyl)porphyrin was completed in 12 hours. The solvent was evaporated and metal complex was purified short silica gel column using dichloromethane and hexane as eluent in 90% yield. ¹H NMR (600 MHz, CDCl₃) δ 7.65 (s, 8H), 1.49 (s, 36H); ¹⁹F NMR (600 MHz, CDCl₃) δ-144.91 (s, 8F).

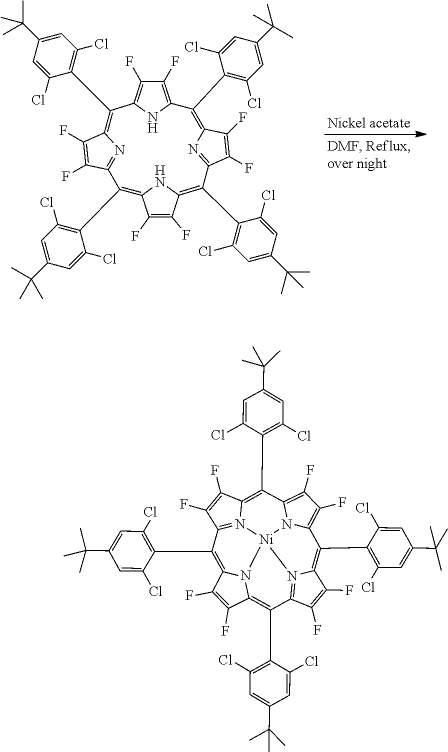

### Preparation of [2,3, 7,8,12,13,17,18-Octafluoro-5,10,15,20-tetrakis(4-(tert-butyl)-2,6-dichlorophenyl)porphinato]platinum

2,3,7,8,12,13,17,18-Octafluoro-5,10,15,20-tetrakis(4-(tert-butyl)-2,6-dichlorophenyl)porphyrin (50 mg) and PtCl₂(DMSO)₂ 2.5 equivalent and sodium acetate 5 equivalent was suspended in 20 mL of chlorobenzene. Reaction mixture was refluxed and progress of reaction was monitored by thin layer chromatography. Metallation of 2,3,7,8,12,13,17,18-octafluoro-5,10,15,20-tetrakis(4-(tert-butyl)-2,6-dichlorophenyl)porphyrin was completed in 8 hours. The solvent was evaporated and metal complex was purified in a short silica gel column using dichloromethane and hexane as eluent in 88%. ¹H NMR (600 MHz, CDCl₃) δ 7.72 (s, 8H), 1.54 (s, 36H); ¹⁹F NMR (600 MHz, CDCl₃) δ-147.00 (s, 8F).

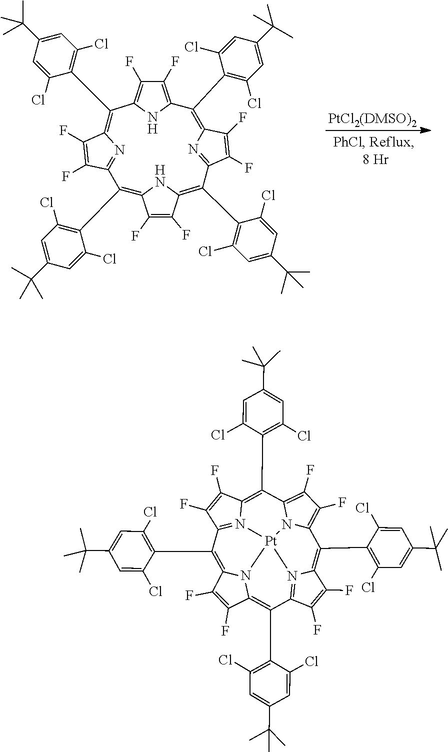

### 2,3, 7,8,12,13,17,18-Octafluoro-5,10,15,20-tetrakis(4-nitrophenyl)porphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

4-Nitrobenzaldehyde 0.932 g (6.17 mM) was added to another round bottom flask equipped with magnetic stirrer bar and diluted with 615 mL of dry dichloromethane. Followed by addition of boron trifluoride diethyl etherate 2.48 mL. 3,4-difluoropyrrole in dichloromethane was added drop wise to reaction mixture over a period of three hours under vigorous stirring. The reaction mixture was stirred for one more hour at room temperature under argon. The progress of reaction is monitored by thin layer chromatography. Reaction was quenched with addition of triethylamine and DDQ were added to oxidize the porphyrinogen. Reaction mixture was stirred for one hour. The reaction mixture was purified by washing with 10 mL of hexane. Polypyrrole was removed by washing the crude product with methanol until the purified 2,3,7,8,12,13,17,18-octafluoro-5,10,15,20-tetrakis(4-nitrophenyl)porphyrin was obtained in 33% yield. 2,3,7,8,12,13,17,18-octafluoro-5,10,15,20-tetrakis(4-nitrophenyl)porphyrin was further purified by crystallization from methanol. The product is characterized by following data: ¹H NMR (600 MHz, CDCl₃) δ 8.62 (d, J=8.6 Hz, 8H), 8.23 (d, J=8.49 Hz, 8H), -4.22 (s, 2H); ¹⁹F NMR (600 MHz, CDCl₃) δ-138.40 (s, 4F), -143.68 (s, 4F); mass: 1.20 g.

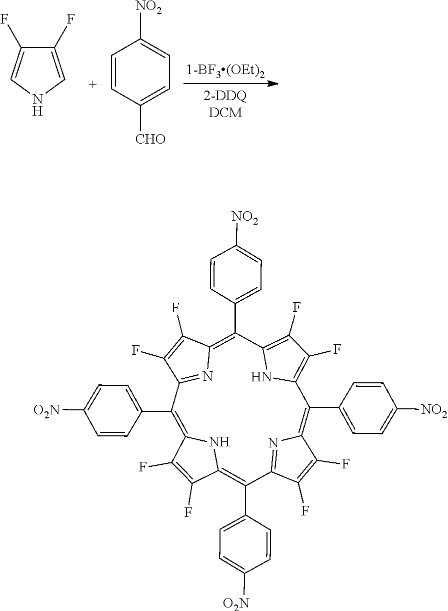 [SMILES: Fc1c[nH]cc1F ; O=Cc1ccc([N+](=O)[O-])cc1 ; O=[N+]([O-])c1ccc(-c2c3nc(c(-c4ccc([N+](=O)[O-])cc4)c4[nH]c(c(F)c4F)c(-c4ccc([N+](=O)[O-])cc4)c4nc(c(-c5ccc([N+](=O)[O-])cc5)c5[nH]c2c(F)c5F)C(F)=C4F)C(F)=C3F)cc1]

### Preparation of 2,3, 7,8,12,13,17,18-Octafluoro-5,10,15,20-tetrakis(3-methyl-4-nitrophenyl)porphyrin

3,3,4,4-Tetrafluoropyrrolidinium chloride 1 g (1) (5.6 mM) was added in a round-bottom and diluted with 5 mL of dry DMSO at room temperature. The reaction mixture was cooled to 0° C. in a cold-water bath, followed by slow addition of potassium tert-butoxide 2.4 g (4.0 equiv) under an argon using powder addition funnel. The mixture was stirred for 40 minutes at room temperature, then cooled to 0° C. and quenched by addition of ice. The reaction mixture was extracted three times with 30 mL of dichloromethane. The combined organic layers were washed four times with water and twice with brine and dried over magnesium sulfate.

3-methyl-4-nitrobenzaldehyde 1.01 g (6.17 mM) was added to another round bottom flask equipped with magnetic stirrer bar and diluted with 615 mL of dry dichloromethane. Followed by addition of boron trifluoride diethyl etherate 2.48 mL. 3,4-difluoropyrrole in dichloromethane was 20 added drop wise to reaction mixture over a period of three hours under vigorous stirring. The reaction mixture was stirred for one more hour at room temperature under argon. The progress of reaction is monitored by thin layer chromatography. Reaction was quenched with addition of triethylamine and DDQ were added to oxidize the porphyrinogen. Reaction mixture was stirred for one hour. The reaction mixture was purified by washing with 10 mL of hexane. Polypyrrole was removed by washing the crude product with methanol until the purified 2,3,7,8,12,13,17,18-octafluoro-5,10,15,20-tetrakis(3-methyl-4-nitrophenyl)porphyrin was obtained in 36% yield. 2,3,7,8,12,13,17,18-octafluoro-5,10,15,20-tetrakis(3-methyl-4-nitrophenyl)porphyrin was further purified by crystallization from methanol. The product is characterized by following data: ¹H NMR (600 MHz, CDCl₃) δ 8.38 (d, J=8.3 Hz, 4H), 8.03 (d, J=8.5 Hz, 8H), 2.86 (s, 12H), -4.26 (s, 2H); ¹⁹F NMR (600 MHz, CDCl₃) δ-138.57 (s, 4F), -143.94 (s, 4F); mass: 1.08 g.

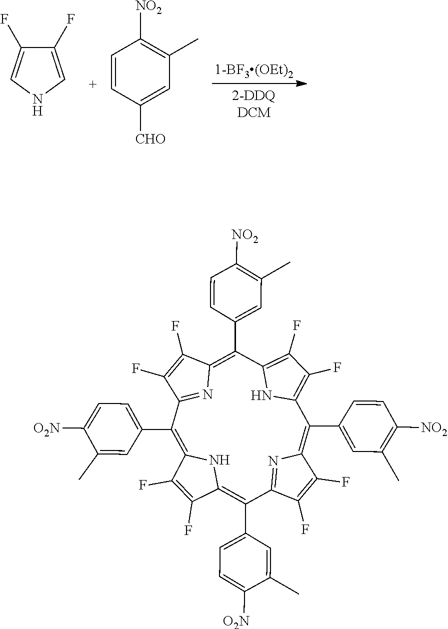 [SMILES: Fc1c[nH]cc1F ; Cc1cc(C=O)ccc1[N+](=O)[O-] ; Cc1cc(-c2c3nc(c(-c4ccc([N+](=O)[O-])c(C)c4)c4[nH]c(c(F)c4F)c(-c4ccc([N+](=O)[O-])c(C)c4)c4nc(c(-c5ccc([N+](=O)[O-])c(C)c5)c5[nH]c2c(F)c5F)C(F)=C4F)C(F)=C3F)ccc1[N+](=O)[O-]]

### Preparation of [2,3, 7,8,12,13,17,18-octafluoro-5,10,15,20-tetrakis(4-nitrophenyl)porphyrinato]zinc

2,3,7,8,12,13,17,18-octafluoro-5,10,15,20-tetrakis(4-nitrophenyl)porphyrin (50 mg) was suspended in 20 mL of dichloromethane and few drops of methanol solution containing a zinc acetate 150 mg was added. Progress of reaction was monitored by thin layer chromatography. Metallation of 2,3,7,8,12,13,17,18-octafluoro-5,10,15,20-tetrakis(4-nitrophenyl)porphyrin was completed in 2 hours. The solvent was evaporated and metal complex was purified in short silica gel column using dichloromethane.

### Preparation of[2,3, 7,8,12,13,17,18-Octafluoro-5,10,15,20-tetrakis(3-methyl-4-nitrophenyl)porphyrinato]zinc

2,3,7,8,12,13,17,18-Octafluoro-5,10,15,20-tetrakis(3-methyl-4-nitrophenyl)porphyrin (50 mg) was suspended in 20 mL of dichloromethane and few drops of methanol solution containing a zinc acetate 150 mg was added. Progress of reaction was monitored by thin layer chromatography. Metallation of 2,3,7,8,12,13,17,18-octafluoro-5,10,15,20-tetrakis(3-methyl-4-nitrophenyl)porphyrin was completed in 2 hours. The solvent was evaporated and metal complex was purified short silica gel column using dichloromethane.

Those skilled in the art will recognize, or be able to ascertain using no more than routine experimentation, many equivalents to the specific embodiments of the invention described herein. Such equivalents are intended to be encompassed by the following claims.

We claim:

1. A method of making a halogenated porphyrin, the method comprising:
(i) reacting a first reactant and a second reactant in a reaction vessel over a period of between 1 hour and 10 hours, optionally under stirring, and forming a first composition, wherein the first reactant and the second reactant are combined slowly over a period of between 1 hour and 10 hours, optionally wherein the first reactant and the second reactant are combined dropwise,
wherein the first reactant is a halogenated pyrrole, a halogenated furan, a halogenated thiophene, or a combination thereof, and the second reactant is an aryl aldehyde or a heteroaryl aldehyde, and
(ii) forming the halogenated porphyrin.

2. The method of claim 1, further comprising after step (i) and before step (ii):
(ia) stirring the first composition over a period of more than one hour, at room temperature, under an inert gas atmosphere, or both.

3. The method of claim 1, wherein the reaction vessel contains a Lewis acid catalyst.

4. The method of claim 1, wherein the reaction vessel contains a complex between an ether and a boron trihalide.

5. The method of claim 1, wherein the reaction vessel contains a boron trihalide dialkyl etherate, optionally boron trifluoride diethyl etherate.

6. The method of claim 2, further comprising after step (ia) and before step (ii):
(ib) quenching the reaction with an organic base, after the formation of a porphyrinogen.

7. The method of claim 6, further comprising:
(ic) adding an oxidizing agent during or after step (ib), oxidizing the porphyrinogen, and obtaining the halogenated porphyrin.

8. The method of claim 1, wherein the halogenated porphyrin is a chiral halogenated porphyrin.

9. The method of claim 1, wherein the halogenated porphyrin comprises bulky substituents at one or more meso-positions of the halogenated porphyrin, wherein the bulky substituents comprise aryl; heteroaryl; alkylaryl; alkylheteroaryl; haloaryl; haloheteroaryl; halo-alkylaryl; halo-alkylheteroaryl; octahydro-1,4:5,8-dimethanoanthracene-9-yl; alkyl [1,1′-biaryl]-4-carboxylate; alkyl [1,1′-biheteroaryl]-4-carboxylate; nitroaryl; alkyl-nitroaryl; or a combination thereof.

10. The method of claim 1, wherein the halogenated porphyrin is a β-fluorinated porphyrin, optionally a β-octafluorinated porphyrin.

11. A halogenated porphyrin having a structure: 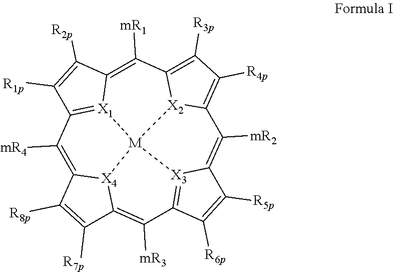
wherein:
M is a transition metal that can be present or absent, and has an oxidation state of 0, +1, +2, +3, +4, +5, +6, or +7,
the dashed lines denote the presence of absence of a bond,
R₁ₚ-R₈ₚ are independently selected from hydrogen, halogen, unsubstituted C₁-C₅ alkyl, substituted C₁-C₅ alkyl, or a combination thereof, wherein at least one of R₁ₚ-R₈ₚ is a halogen, mR₁-mR₄ are independently selected from aryl, heteroaryl, alkylaryl; alkylheteroaryl; haloaryl; haloheteroaryl; halo-alkylaryl; halo-alkylheteroaryl; octahydro-1,4:5,8-dimethanoanthracene-9-yl; alkyl [1,1′-biaryl]-4-carboxylate; alkyl [1,1′-biheteroaryl]-4-carboxylate; nitroaryl; alkyl-nitroaryl, or a combination thereof, wherein mR₁-mR₄ are not all aryl,
X₁-X₄ are independently nitrogen, oxygen, or sulfur, preferably nitrogen, and X₁-X₄ independently contain zero, one, or two hydrogen atoms according to valency, and mR₁-mR₄ are optionally substituted with one or more substituents.

12. The halogenated porphyrin of claim 11, wherein mR₁-mR₄ are independently selected from aryl, alkylaryl; haloaryl; halo-alkylaryl; octahydro-1,4:5,8-dimethanoanthracene-9-yl; alkyl [1,1′-biaryl]-4-carboxylate; nitroaryl; alkyl-nitroaryl; or a combination thereof.

13. The halogenated porphyrin of claim 11, wherein mR₁-mR₄ are independently selected from aryl, heteroaryl, 2,4,6-trialkylaryl; 2,4,6-trialkylheteroaryl; 2,6-dihaloaryl; 2,6-dihaloheteroaryl; 2,6-dihalo-4-alkylaryl; 2,6-dihalo-4-alkylheteroaryl; octahydro-1,4:5,8-dimethanoanthracene-9-yl; alkyl [1,1′-biaryl]-4-carboxylate; alkyl [1,1′-biheteroaryl]-4-carboxylate; 4-nitroaryl; 3-alkyl-4-nitroaryl; or a combination thereof.

14. The halogenated porphyrin of claim 11, wherein at least one of R₁ₚ-R₈ₚ is fluorine.

15. The halogenated porphyrin of claim 11, wherein R₁ₚ-R₈ₚ are fluorine.

16. The halogenated porphyrin of claim 11, wherein M is present, the dashed lines denote the presence of a bond, and M is a first-row transition metal, a second-row transition metal, or a third-row transition metal.

17. The halogenated porphyrin of claim 11, wherein M is a zinc, a nickel, or a platinum.

18. The halogenated porphyrin of claim 11, wherein M is absent.

19. A halogenated porphyrin, having a structure: 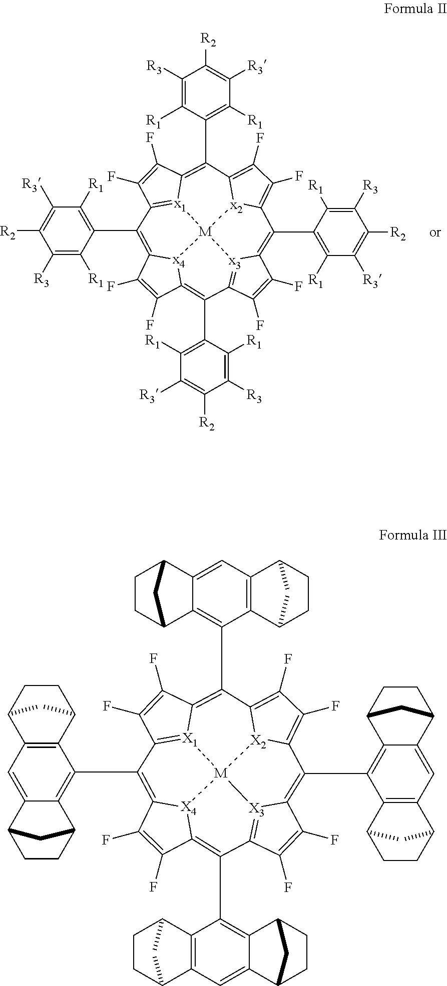
wherein:
for Formula II and Formula III, M is a transition metal that can be present or absent, and has an oxidation state of 0, +1, +2, +3, +4, +5, +6, or +7,
the dashed lines denote the presence of absence of a bond,
X₁-X₄ are nitrogen, and X₁-X₄ independently contain zero, one, or two hydrogen atoms according to valency,
for Formula II:
R₁, R₂, R₃, R₃′=H;
R₁, R₂=—CH₃, R₃, R₃′=H;
R₁=Cl, R₂ R₃, R₃′=H;
R₁=Cl, R₂=—CH₃, R₃, R₃′=H;
R₁=Cl, R₂=-tert-butyl, R₃, R₃=H;
R₁, R₃, R₃′=H, R₂=Br;
R₁=Br, R₂, R₃, R₃′=H;
R₁=Br, R₂=-tert-butyl, R₃, R₃=H;
R₁, R₃, R₃′=H, R₂= 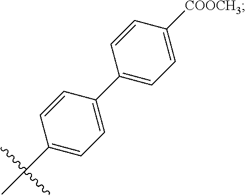 [SMILES: *c1ccc(-c2ccc(C(=O)OC)cc2)cc1]
R₁, R₃, R₃′=H, R₂=—NO₂; or
R₁, R₃, =H, R₂=—NO₂, R₃′=—CH₃.

20. A composition comprising the halogenated porphyrin of claim 11.
